(19) United States
(12) Patent Application Publication
(10) Pub. No.: US 2007/0077615 A1
(43) Pub. Date: Apr. 5, 2007

(54) Methods and apparatus for automated spore-culturing and monitoring

(75) Inventor: John R. Gillis, Bozeman, MT (US)

Correspondence Address: Raymond N. Baker;SHANLEY & BAKER, P.L.L.C., P.O. Box 9995, Washington, DC, 20016-8995, US

(73) Assignee: SGM Biotech, Inc., Bozeman, MT (US)

(21) Appl. No.: 11/542,221

(22) Filed: Oct. 4, 2006

Related U.S. Application Data
(60) Provisional application No. 60/723,672, filed on Oct. 5, 2005.

(51) Int. Cl. C12Q 1/18; C12M 1/34; C12Q 1/22

(52) U.S. Cl. 435/31; 435/32; 435/287.1

(57) ABSTRACT

Processes and apparatus for functioning, subsequent to exposure to a designated microbial-biocidal treatment-cycle, for automated measurement, reporting, and recording, along with treatment-cycle data, for verifying effectiveness of each such treatment-cycle, by automatically monitoring a respective biological-indication (B-I) Test Ampoule for such a cycle. Prior burdensome requirements on personnel, in establishing and evaluating bacterial-lethality, and documenting all relevant steps and data are eliminated; while enabling accurate and prompt evaluations of bacterial-lethality. B-I Test-Ampoules types having differing sizes and configurations are evaluated in respective housing-structure test-cell receptacles, each having a correlated size and configuration with that of the respective Test-Ampoule; which augments establishing the culturing temperature required for the B-I Test Ampoule being evaluated. Further, evaluation of a treatment-cycle failure can be expedited by use of either a Colormetric Technology or Spectroscopic Technology radiant-energy embodiment to determine surviving microbe, if any, activity within about three to five hours after initiating culturing testing, in place of the usual time of about forty hours for completing evaluation without these technologies.

### RELATED APPLICATION

This application claims the benefit of U.S. Provisional Patent Application Ser. No. 60/723,672 filed Oct. 5, 2005.

### INTRODUCTION

This invention relates to automated procedures for monitoring sterility test-indicators individually for results following intended exposure of a test-ampoule to a selected microbial-biocidal treatment cycle. More particularly, this invention is concerned with providing methods and apparatus capable of automated analyses, following intended exposure to biocidal treatment cycle of individual test-ampoules capable of providing biological-indication (B-I) of microbial status while, also, providing automated-recording of documentation of microbial-biocidal treatment-cycle information and measurement data, for verifying biocidal-effectiveness on each such automatically-monitored test-ampoule, along with data supporting results of each respective B-I Test-Ampoule evaluation.

### OBJECTS OF THE INVENTION

Primary objects are
(i) enabling accurate and prompt evaluation of constituents, within a sealed test-ampoule, for providing a biological-indication (B-I) of bacterial-lethality; by
(ii) determining the presence or absence of microbial-activity within such a sealed test-ampoule, and
(iii) providing micro-processor control of documented observations in a printed record of respective individual incubation steps, timing, pertinent measured biocidal data, or absence thereof, relating to each respective microbial-biocidal treatment-cycle.

A related object provides for automated carrying-out of a plurality of individual-procedures controlling timing aspects, measurements, and recordation of data of intended procedures, without requiring an operator to: watch for, individually-manage, or personally determine and record data necessary for properly monitoring and reporting results of individual microbial-biocidal treatment-cycles, on respective individual test-ampoules.

A further related object is to provide housing-structure embodiments with controllable-heating means for individual test-ampoules from one, or from a plurality of intended microbial-biocidal treatment cycles.

Another related object for housing-structures is providing for differing-configurational and sized test-ampoules to be efficiently utilized and evaluated, with each providing a biological evaluation of its respective microbial-biocidal treatment cycle.

An inter-related object enables combining observational and functional equipment within a selected housing-structure by correlating the configuration and size of an individual test-ampoule, with those of a housing-structure test-cell (receptacle) so as to facilitate and augment achievement of culturing-conditions for analyzing a microbial-biocidal treatment-cycle.

Further specific objects for significantly-decreasing expected times for B-I evaluations of individual Test-Ampoules are provided by utilizing selected radiant-energy-source means and selected electrometric indicator means, for expediting evaluation of microbial-status within a liquid nutrient-growth medium (NGM) which remains sealed within an individual B-I Test-Ampoule.

An added object is to provide readily-observable visual and/or auditory alarm-type indication of the status of an individually-monitored test-ampoule providing for prevention of early release of goods which have not progressed to achievement of desired microbial-biocidal standards, during production operations.

Other objects and contributions of the invention as claimed are disclosed in conjunction with the following description in combination with the accompanying drawings.

### BRIEF DESCRIPTION OF THE DRAWINGS

FIG. 1 is a top-plan view of a housing-structure of the invention for describing entry locations, for individual B-I Test-Ampoules, into respective test-cells for establishing culturing conditions and carrying-out certain monitoring steps, while also providing entry locations for certain initial steps, in accordance with the invention.

FIG. 2 is an elevational schematic cross-sectional partial view of controllable heating-block means of the invention for describing differing size and configuration housing-structure test-cell receptacles for individually receiving a B-I Test Ampoule of correlated-configuration for augmenting evaluation steps of the invention, subsequent to exposure of a B-I Test-Ampoule to a selected microbial-biocidal treatment-cycle.

FIG. 3 is an elevational view of several types of individual B-I Test-Ampoules for interfitting, in accordance with the invention, within a respective selected test-cell of correlated size and configuration, so as to facilitate establishing culturing-conditions for biological evaluations of the invention.

FIG. 4 is an enlarged schematic presentation for describing interacting functional components arranged with respect to an individual-cell with Test-Ampoule in place, for decreasing the time required, in accordance with the invention, for evaluating the microbial-status of such B-I Test-Ampoule.

FIG. 5 is a schematic overall arrangement of test apparatus of the invention presenting electrical and electronic circuit means for presenting prompt monitoring reporting, and recording function equipment, including prompt radiant-energy analyses of designated B-I Test Ampoules.

FIG. 6 is a schematic flow-chart for describing procedural steps in carrying out microbial-status evaluations of the invention.

FIG. 7 is a record format, completed in accordance with the invention, for identifying a microbial-biocidal treatment-cycle, relevant data, and biocidal results of carrying-out intended operations on a specific B-I Test-Ampoule.

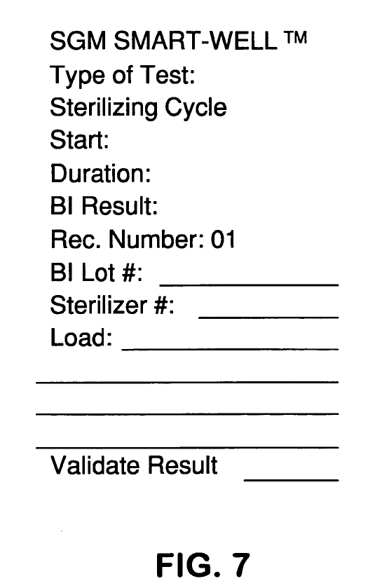

### DETAILED DESCRIPTION

Microbial-biocidal treatment-cycles, used by hospitals rely on various methods and sterilants; for example: (i) heating utilizing saturated-steam and other means for thermal-processing, (ii) microbe-destructive gases, such as ethylene oxide (ETO), and (iii) carboxide combinations; as well as other sterilants for destruction of infectious bacteria. Microbial-biocidal treatment cycles used by batch-food processors, as part of preparation for non-refrigerated marketing, rely largely on thermal-processing for destruction of food-spoilage bacteria. This invention is concerned with the work remaining, when any such selected microbial-biocidal treatment-cycle has been completed, for evaluation of treatment-cycle effectiveness, or lack thereof, in achieving bacterial-lethality.

Biological Indicator (B-I) Test-Ampoules rely on establishing microbial-culturing conditions for making microbial-biocidal evaluations. B-I evaluations provide basic, comprehensive, and reliably-accurate evaluations; and, are preferred, for example, by the Food & Drug Administration (FDA). The invention provides evaluations of microbial growth, or absence thereof, while automating methods and means for individually monitoring microbial-biocidal effectiveness in one or in a plurality of individual types B-I Test-Ampoules.

By providing culturing-conditions for designated types of treatment-cycles and enabling evaluating specific B-I Test-Ampoule types, for respective biocidal-treatment cycles, the invention provides for analyzing various sizes and configurations of B-I Test-Ampoules. A B-I Test-Ampoule for use in the invention contains: (i) selected bacteria which are relevant to a designated selected treatment-cycle and its selected sterilant, (ii) a liquid nutrient-growth-medium (NGM) for culturing bacteria, if any survive the treatment-cycle, and (iii) means for automated monitoring, indicating, and recorded verification of the presence or absence of microbial-activity, subsequent to usage in an intended biocidal treatment-cycle.

Monitoring such results in B-I Test Ampoules effectively involves a plurality of carefully-executed steps starting with selecting and establishing culturing conditions for a particular cycle and/or sterilant; plus, requirements for documented-recording of each test step, along with its timing and respective measured results. Executing such individual test requirements is time-consuming, demanding, and imposes restraints for increasing accuracy.

The present invention decreases certain prior burdensome requirements in evaluating bacterial-lethality, by establishing and implementing crucial standards for automated test-verifications and record-keeping; while, diminishing individual bookkeeping burdens on personnel by cycle-identification automated timing of individual steps and data-collecting events for verification, reporting and recording purposes.

Present concepts for automating microbial-biocidal evaluations significantly decrease burdens on practitioners while enabling accurate, prompt, and reliable evaluations of bacteria-lethality; plus, establish authenticated documentation of times and recorded results. Technological important contributions of the invention involve assembly and functioning of automated culturing and monitoring equipment which not only improve accuracy; but, also, enable prompt evaluations of microbial-status. In that regard, it should be noted that “health-wise” a properly-executed prompt biocidal-evaluation failure of a microbial-biocidal treatment-cycle becomes significant because of otherwise dire results. That is, preventing release of contaminated-goods which can sometimes occur due to short-supplies of items properly exposed to bacterial-lethality which can occur for certain surgical instruments which are in short supply in a medical facility. Also, prompt detection of a failure in a food treatment-cycle is important in preventing distribution, for non-refrigerated marketing following inadequate biocidal treatment of such foods.

The present invention automates: culturing, testing, and basic steps for verification of desired bacterial-lethality; plus, automatically prepares authenticated verification records for protecting practitioners; and, ultimately, benefitting users of treated goods. The invention enables handling and testing of a several types and sizes of B-I Test-Ampoules; for example, by sub-dividing an individual housing-structure to provide incubation for more than that one time of test-ampoules; while, also, providing housing-structure embodiments with increased numbers of cells selected so as to meet testing and/or shipping requirements for large organizations.

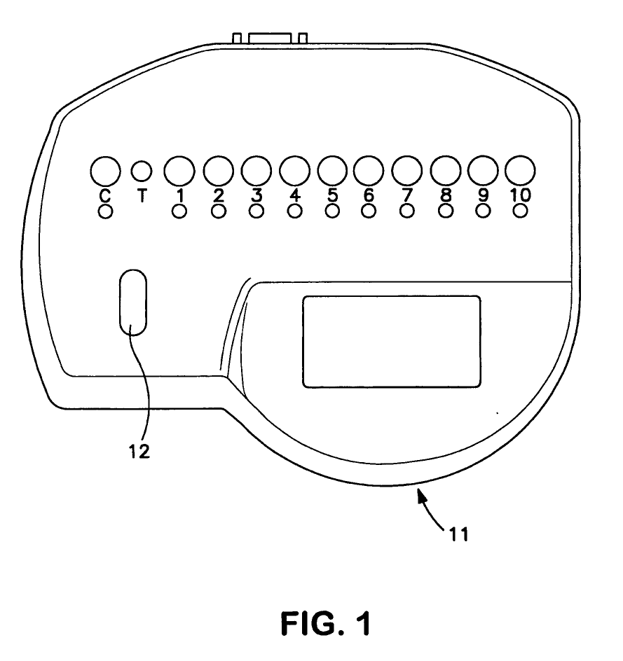

FIGS. 1, 2, and 3 are referred to herein for summarizing descriptions of differing sizes and differing configurations of both test-cell receptacles and test-ampoules; and, for describing housing-structure (i) functions, plus: (ii) capabilities for handling differing configuration sized and B-I Test-Ampoules, and (iii) facilitating testing by correlating differently-sized and differing-configuration test-ampoules with differently sized and differing-configuration test-cells.

Such coordinating combination, as described herein, is particularly helpful with increased-size housing-structure for a single type of Test-Ampoule, especially for use by large business organizations.

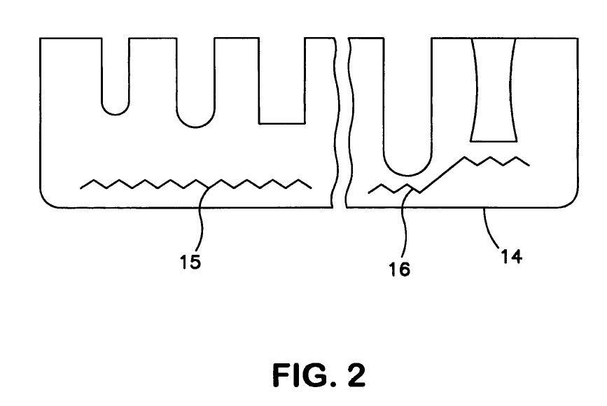

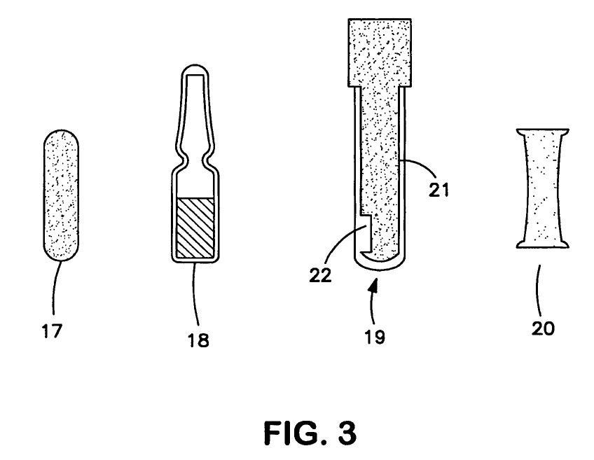

However, FIGS. 1, 2, and 3 are helpful in summarizing differences in test-ampoules and test-cells. The controllable heating means of FIG. 2, shows differently sized and configurational receptacles, provided in a single housing-structure for describing differing test-ampoules. Also, differing sizes and configurations for B-I Test-Ampoule types are shown in FIG. 3. Correlating structural-characteristics of test-ampoules with respective structural-characteristics of a test-cell contributes to decreasing time for achieving a “culturing” temperature requirement; which increases evaluations of B-I Test-Ampoules, and related measurements thereof for evaluating of bacterial lethality, as disclosed herein.

Individual housing-structures are made available with selected numbers of cells as well as specialized test capabilities, determined largely by the cycle and sterilant used for microbial-biocidal treatment. That is, providing individual housing-structures for designated treatment-cycles using designated sterilants, facilitates handling of large numbers of designated B-I Test-Ampoules in a single housing-structure operated to maintain required culturing conditions. Variously-sized housing-structure embodiments with ten, fifty, or as many as one hundred, test-cells enable specifying a housing-structure embodiment for a designated treatment cycle utilizing a designated sterilant; which increases the capability for timely meeting volume requirements of larger enterprises.

The exterior top-plan view of FIG. 1 presents a housing-structure for describing step and measurements utilizing differing types of individual B-I Test-Ampoules. Respective test-cells are numerically-designated sequentially (one through ten) in housing structure 11 of FIG. 1; each of those test-receptacles accommodates one test-ampoule. As mentioned, preparing housing-structure embodiments for a single designated type of test-ampoule, enables timely evaluations of greater numbers of test-ampoules, as may be required by larger organizations. And, enables a housing-structure embodiment to be designed for designated culturing conditions of designated B-I Test-Ampoules; in that way, differing-culturing-temperature requirements can be provided by selecting a single housing-structure embodiment, which is manufactured for meeting those requirements, where and when needed.

Housing-structure 11 of FIG. 1 also presents openings marked “C” and “T”, which are located for starting use of a selected housing-structure embodiment. The “C” location provides for initially verifying the overall working-operation of the housing-structure in uncovering faults. A tubular container, which contains live-bacteria, is inserted into opening “C” for verifying proper overall operation of the housing-structure, for fault-finding evaluation purposes, before starting to receive designated-type(s) of recently-exposed test-ampoules. The opening labeled “T” contributes to versatile operations by enabling currently verifying that a desired operational culturing-temperature, for designated tests-ampoules to be analyzed and their perspective treatment-cycle, has been established utilizing a controllable heating-block of the invention; for example, as specified for a selected housing-structure embodiment handling requirements for a particular-type of B-I Test-Ampoule.

An added opening 12 shown in the FIG. 1 top-plan view of housing structure 11, is provided for describing function, which can be performed when needed. That is, for release of a liquid-nutrient growth medium (NGM) when necessary for a particular type of B-I Test-Ampoule. That particular type of B-I Test-Ampoule, as later described in more detail, requires rupture of an internally-located separately-sealed capsule for release of a liquid (NGM) for culturing live bacteria, if any survive a treatment cycle. FIGS. 1-3 are for facilitating description of several types of test-ampoules and test cells; greater numbers of which could be provided in a single selected housing-structure embodiment, as described above.

Certain types of B-I Test-Ampoules require release of a separately-internally-sealed liquid NGM when needed, that can be accomplished by providing a housing-structure embodiment, with an opening for rupture of such an internally-disposed sealed capsule of liquid-nutrient-growth-medium (NGM). Such rupture and release of such separately-sealed NGM are accomplished by insertion, of that type of test ampoule, into an elongated opening, such as 12 of FIG. 1. Directed movement within that elongated opening, ruptures such a frangible sealed-internal-capsule, releasing that confined “NGM” for purposes of contacting, and culturing, surviving microbes, if any.

Controllable heating-block 14, which is shown schematically in FIG. 2, is located within housing-structure 11. Heating-block 14 is preferably formed from a heat conductive material, such as, a not-readily-corrodible metal. Heating-block 14 of FIG. 2 is used for helping to illustrate, and describe, concepts for decreasing the time required to reach a required culturing temperature. That is accomplished by correlating size and configuration of a test-cell, with the size and configuration of a specific type of B-I Test-Ampoule. Housing-structure embodiments of the invention can be structured for a single specific type of test-ampoule, requiring a specific heat-treatment cycle; which requirement may, for example, be determined by the sterilant used and/or temperature requirements.

Several differing-configurational receptacles, selected from those available, are shown in FIG. 2 for describing several differing individual types of B-I Test-Ampoules which can be accommodated. Individual test-cell receptacles of housing-structure 11 are manufactured to provide a correlated-fit, with a matching configuration and sized B-I Test-Ampoule to be evaluated. Such correlated-fit concepts contribute significantly to expediting establishment of culturing-temperature; which is relied-on and automatically-monitored for carrying-out the invention; in which Biological-Indicator (B-I) Test-Ampoules are utilized to determine the presence, or absence, of microbial-activity within an individual B-I Test-Ampoule, rather than merely determining that a specific temperature was achieved.

FIG. 2 schematically presents sets of heating-wire elements, for establishing a designated culturing temperature for contiguous B-I Test Ampoules, as placed for accommodating particular illustrated types of B-I Test-Ampoules, each occupying a contiguous receptacle. However, in planning and manufacturing more efficient housing-structure embodiments, it is preferable to provide for increased numbers of configurationally-similar receptacles which utilize the same designated culturing conditions for a single heat treatment-cycle.

Culturing temperatures can vary; for example: test-ampoules from ethylene-oxide (ETO) cycles, utilize a temperature of about 35° C. to 39° C.; while those from saturated-steam cycle types, use a temperature at about 55° C. to 60° C. Housing structure embodiments made practical by the invention, enable utilizing numerically-large numbers of substantially-identical test-ampoules for a designated treatment-cycle; so as accommodate large numbers of B-I Test-Ampoules selected, for example, a single treatment-cycle which utilizes a single culturing temperature for B-I evaluation purposes.

FIG. 3 is a schematic view, in cross-section, for describing several differing types of test-ampoules, each capable of providing a biological-indication (B-I) of microbial status. The test-ampoules shown in FIG. 3 do not exhaust the possible configurations for B-I Test-Ampoules. Configurationally-different Test-Ampoule types can be accommodated, individually, in a test-cell having a correlated size and configuration with such a Test-Ampoule; such correlation facilitates heat-transfer to liquid nutrient growth medium (NGM) within a respective B-I Test-Ampoule type.

Other configurations, than those specifically-shown in FIG. 3, continue to be developed for B-I Test-Ampoules; and, such developing type(s) can readily be utilized by following present teachings which provide for proper correlation in size and configuration, for a test ampoule, in relation to that of the test-cell receptacle being used. Augmenting achievement of a desired rate of heating for establishing the desired culturing temperature, within a B-I Test-Ampoule, is an important objective. More specifically, augmenting the rate of heating can decrease the time for evaluation of biocidal-status. Also, present evaluations can be expedited utilizing transmitted radiant-energy; radiant-energy technology teachings of the invention are described in greater detail later herein.

Each B-I Test-Ampoule of FIG. 3 holds a liquid nutrient-growth-medium (NGM) for supporting microbial-action should any of the bacteria, selected for a test-ampoule, survive exposure to a designated microbial-biocidal treatment-cycle. Test-Ampoule 17 is a compact self-contained biological-indicator which is designated largely for monitoring industrial steam sterilization of liquids; and, is marketed by Applicant as the SterilAmp® Test-Ampoule, U.S. Registration No. 1,660,494. In the SterilAmp® B-I Test-Ampoule, the selected bacteria are in contact with the NGM during storage; therefore, low-temperature storage of such test-ampoules is utilized, prior to exposure in a selected microbial-biocidal treatment-cycle.

Test-Ampoule 18 of FIG. 3 is a type used largely for testing bottled-liquids for hospital usage; it is designated by Applicant as MAGNAAmp®, U.S. Registration No. 2,551,584. In respective test-ampoules 17 and 18, the culturing medium of each is in contact with the microbes during low-temperature storage prior to usage; and, during respective selected microbial-biocidal treatment-cycles. Neither such self contained (B-I) Test-Ampoule, requires rupturing of an internal capsule to release a nutrient-growth-medium (NGM) for completing evaluation of the selected microbial-biocidal treatment-cycle. The incubation temperature for selected saturated-steam through processing cycles can be in the range of 55° C. to 60° C. The prior required incubation-time, for proper evaluation of those cycles, has been at least about forty-eight hours. However, radiant-energy methods and means, described later herein, significantly decrease the time required for a B-I evaluation of the microbial-status of any of the various types of test-ampoules described.

In a B-I Test-Ampoule of the type designated 19 in FIG. 3, liquid NGM 21 is held within a separately-sealed internal-capsule; held within an external polymeric container which includes a special polymeric cap; permitting access of sterilant gas while preventing escape of functional operating contents. The liquid nutrient growth medium (NGM) of Test-Ampoule 19 is not in contact with the selected bacteria during the designated microbial-biocidal Treatment-Cycle. Those bacteria are located on strip 22, which is within the polymeric exterior-container which encapsulates the internal-capsule within of the B-I Test-Ampoule 19. Note that the liquid Nutrient-Growth-Medium (NGM) 21, is not in contact with the bacteria on strip 22, prior to fracture of the frangible sealed-inner-capsule, which is held within the exterior-container. That exterior polymeric container is sufficiently-pliable so as to enable rupture, of the frangible sealed-inner-capsule, by mechanical pressing forces through the pliable-polymeric wall of the exterior container.

B-I Test-Ampoule 19 is marketed by Applicant under the trademark E-Z Test®, U.S. Registration No. 1,647,985. As noted, strip 22 is not in contact with the NGM 21 during a selected microbial-biocidal treatment-cycle. Any microbes on strip 22 which survive the selected biocidal-treatment cycle are first brought into contact with liquid NGM 21 by rupturing the described frangible-interior capsule. That rupturing action within the polymeric external container can be carried-out, for example, within the slotted-opening 12 shown in FIG. 1; and, takes place following completion of the treatment-cycle, before placement of the Test-Ampoule; within correlated-configuration test-cell receptacle, for initiating the culturing-conditions for B-I evaluation of bacteria-lethality.

For testing batch-processed and packaged foods for non-refrigerated marketing, test-ampoule 20, as shown in FIG. 3, is fabricated from thin-polymeric-sheet materials. Enabling fabrication of pliable test ampoules which are selectively-located, individually, during processing of such foods, in so-called “monitoring” containers; those are specifically-located within a longitudinally-extended production processing line. Test-ampoules within bracketing pairs of those monitoring-containers, when analyzed, characterize the status of intermediately-located “associated”-containers in the production line. These procedures are described in more detail in Applicant's copending patent application, U.S. Ser. No. 11/410,196 “EVALUATING BACTERIAL LETHALITY OF CONTAINERIZED FOOD PRODUCTION”; which is included herein by reference.

Flexible polymeric-sheet B-I Test-Ampoules are used in batch processing and packaging foods for non-refrigerated marketing, under the mark STERIL-FLEX™; as shown in Applicant's U.S. Trademark application Ser. No. 76/657,610. When used in automated incubation and testing equipment of the present application, a STERIL-FLEXT™ B-I Test-Ampoule is placed within a thin semi-rigid-polymeric holder approximately the shape of the STERIL-FLEXT™ B-I Test-Ampoule; which enables placement in a correlated size and configuration test-cell receptacle, for augmenting heating to culturing conditions for evaluation of bacterial-lethality, as disclosed herein.

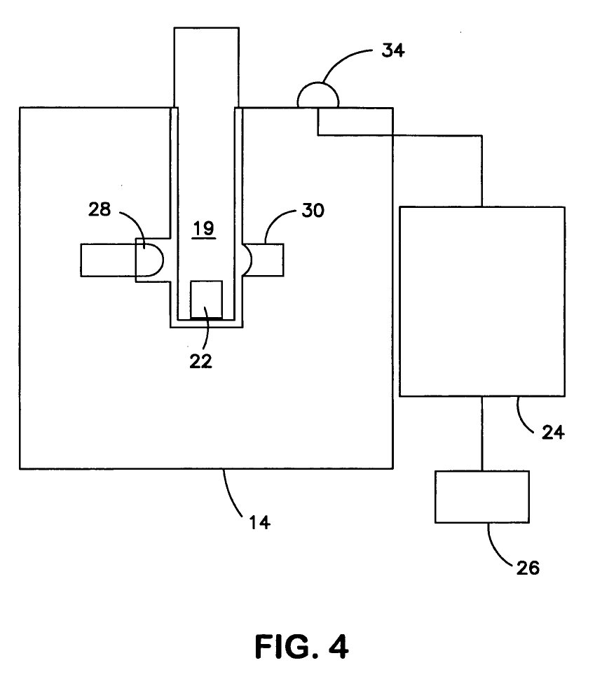

The schematic arrangement of FIG. 4 can be adapted for describing placement of colormetric radiant-energy source and detector means; plus other functioning components, which are interrelated with respect to each other, for decreasing the time required for detecting a treatment-cycle failure, within a B-I Test-Ampoule, as held within an individual test-receptacle. Those components function to monitor: (i) time and temperature when initiating culturing-conditions, (ii) times and temperature for all measurements while, also, (iii) rendering an early “alert-type” visible and/or audible notification of results, to a practitioner utilizing the schematically-represented components of FIG. 4; plus accurate documented recording of: timing for (a) each function, and (b) providing recording of relevant data supporting a B-I evaluation as made. An important advantage of expedited evaluation of treatment-cycle failure, is that it enables prompt interruption before planned usage, or delivery, of instruments for use; or foods for marketing.

In practice of the invention, housing-structure embodiment arrangements and culturing-conditions are selectively established considering a number of factors; such as: the microbial-biocidal treatment cycle, the type of sterilant used, and type(s) of B-I Test-Ampoules to be used. Establishing a culturing temperature uniformly throughout a housing-structure embodiment enables eliminating any requirement for establishing differing culturing temperatures, for differing types of B-I Test-Ampoules within various test-receptacles of a housing-structure.

As mentioned in describing FIGS. 1-3, correlation of size and configuration of a test-ampoule within a respective test-receptacle can be handled within a single housing-structure. Enabling, increasing the number of an identified type of B-I Test-Ampoule, within each test-cell to facilitate meeting requirements of a large organization. Heating takes into account a specifically-selected bacteria culturing temperature following use of a particular type of treatment-cycle to be evaluated.

Batch-food processing STERIL-FLEX™ test-ampoules would use a thermal-processing temperature which is determined by selected food-spoilage bacteria and by the pH of the food(s) being processed; as described in the above-referenced U.S. application Ser. No. 11/410,196 which is included herein by reference. In both instances the number of test cells in the housing structure can be increased for purposes of increasing production.

As indicated by schematic presentation in FIG. 4, certain functioning components are located within of housing-structure embodiment; and, other components are inter-connected; but, are located externally of that housing-structure. A microprocessor for each radiant-energy analysis embodiment, is connected to measuring-components within the housing-structure for reporting, signaling and recording purposes; as well as, for radiant-energy analyses.

Relevant information, such as: the culturing time, the change, if any, occurring in the liquid NGM, and, recording of all respective data, are directed and handled by microprocessor 24 in FIG. 4. The record for each procedure, and the final evaluation, are maintained for each activated receptacle by printer 26; which also can be located externally of the housing-structure, as shown. A test-cell receptacle for each B-I Test-Ampoule, and its heating means, are located within the housing-structure; and, all measuring equipment is wired for functioning with each respective test-cell receptacle; and, expedited radiant energy analyses, enables decreasing the overall number of test-cells in a housing-structure embodiment.

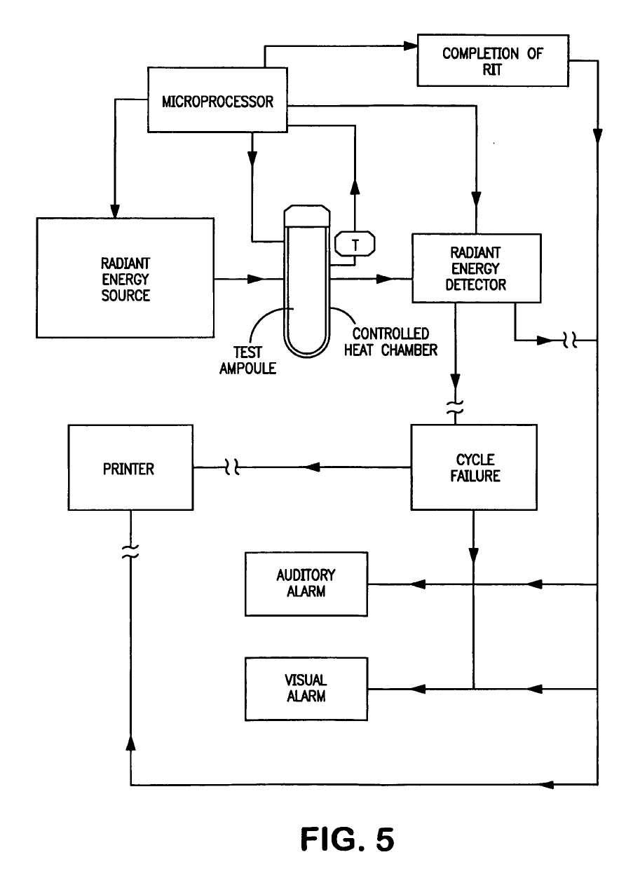

Automated-monitoring equipment, as described in relation to FIGS. 4 and 5, enables the expedited evaluations of the present invention, by utilizing “radiant-energy” analyses. As taught herein, prompt radiant-energy analyses are particularly germane and important for prompt B-I Test Ampoule indications of failure of a treatment cycle. Colormetric-technology analysis and/or the selected Spectroscopic-technology analysis each provide for such a prompt evaluation.

The equipment as schematically presented in FIG. 4 will be first described for prompt colormetric-technology analysis (alarm-type notice of treatment-cycle failure). Use of “colorimetry-technology” for prompt “radiant-energy” analysis has at least one specific advantage; that is: (i) selection from a wider range of color-producing pH indicator components is made available; (ii) readily-understandable steps are also made available for carrying out such operations, and (iii) ready correlation of observations for reaching conclusions. A colormetric type of radiant-energy analysis of the invention is described in more detail in the following presentation.

Referring to FIG. 4, light emitting diode (LED) 28, is selected to project a prescribed frequency of visible light; for example: so as to emit a beam having a distinct “yellow” color. That light is beamed for passage through the liquid NGM of a Test-Ampoule for making a B-I evaluation. In the example of FIG. 4, the released liquid NGM is within a B-I Test-Ampoule, such as 19, which was described in relation to FIG. 3. In evaluating a sterilizing cycle using B-I Test-Ampoule 19, any surviving bacteria on strip 22 come into contact with the liquid nutrient growth medium (NGM) upon rupture of such sealed inner-capsule. The integrity, of all such capsule constituents is maintained within the exterior polymeric container preventing any misleading contamination, before or subsequent to fracturing the inner sealed capsule as carried out for purposes of initiating culturing of live bacteria, if any.

In a colormetric-technology prompt-indication embodiment of the invention, the color of the liquid NGM in a Test-Ampoule is determined based on selection of a particular “pH indicator”, as described below.

A liquid nutrient growth medium (NGM) for biological-indication (B-I) is preferably selected to include the following constituents, or their equivalents:

| Constituent | Grams/liter |
|---|---|
| Glucose | 5.0 |
| Tryptone | 8.5 |
| Soytone | 10.0 |
| Soluble Starch | 1.0 |
| Yeast Extract | 0.5 |

In accordance with “colormetric” analysis teachings of the invention, a pH indicator is selected for purposes of precoloring the liquid NGM released from a B-I Test-Ampoule. Pre-selecting such a color, depends on such selection of a “pH Indicator” constituent for the NGM; e.g.:
(i) Bromocresol Purple, for thermal-processing cycles using specifically designated bacteria, establishes a purple color;
(ii) Phenol Red for certain types of cycles and designated bacteria, establishes a red-color, and
(iii) Bromothymol Blue, can be selected for other examples, which rely on using differing wave-length light to be projected from LED 28 of FIG. 4.

If any microbes, within such B-I Test-Ampoule 19, survive the biocidal-treatment cycle being tested, such microbes will produce other cells or spores in the liquid NGM. That metabolic action is acidic, which changes the color of the liquid NGM provided by the selected pH indicator. That is, growth of bacterial cells, or spores, provides a change in the acid level of the NGM. The result is that the selected color of the liquid NGM changes due to such change in pH level. And, more specifically, that resultant change in pH effects the color capable of being transmitted by the liquid NGM.

When Bromocresol Purple is used as the pH indicator, the color of the liquid NGM is purple. The color selected to be emitted by LED 28 is a distinct yellow visible-light. That distinct-yellow visible light will not be transmitted by such purple NGM is established by the pH indicator. However, acidic change responsive to “microbial-action” can occur as a result of establishing culturing conditions, if any microbes survive the treatment-cycle. But, resultant initial changes would not be ascertainable by the naked eye, in less than about forty-eight (48) hours of such acidic action.

However, radiant-energy analysis of the invention, detects minimal transmittances of such “tell-tale” yellow in the NGM the latter are readily detected by photo-detector (PD) 30 which is responsive to the yellow-wavelength. And, microprocessor 24 records that such microbial-action is taking place in the NGM, in recorder 26, as well as recording the time.

In addition, present teachings provide for an “alarm-type” report, to be promptly and clearly available to a user or operator.

In the example being described, when a test-ampoule is initially placed into its respective test-cell, status-light 34, associated with the receptacle holding the B-I Test-Ampoule would display the color yellow; meaning that the procedural operation is beginning; microprocessor 24 would record the time and start the incubation timer. If bacteria are incubated, status-light 34 of FIG. 4 would blink “red”, rather than yellow, under control of microprocessor 24; and, similarly, an audible alarm could also be utilized.

Results thus obtained when using colorimetry-technology analysis, are readily observable and comprehended by operating personnel. Such a colormetric-technology evaluation is available, within about three to about five hours of establishing culturing conditions; that is, by detecting a response to a minor streak of the “tell-tale yellow” being-transmitted. That improved-timing finding, indicating failure of the respective treatment-cycle, has very-helpful and many satisfactory results for many installations, in place of a forty-eight (48) hour, or more requirement when not using a radiant-energy technology analysis, as taught herein.

In practice of the colormetric-technology of the invention, the color of the selected pH indicator for the NGM, acts as a blocking “filter”. For example, when a Bromocresol Purple color exists in the NGM, it will not transmit other colors effectively blocking any incremental streaks of such yellow light. Other pH indicators, as named, can be used by selecting differing light wavelengths for LED 28 and (photo-detector) PD 30.

In the colormetric embodiment being described, PD 30 responds only to the transmitted “yellow” light. Therefore a change within the NGM, which enables incremental tell-tale yellow light from LED 28 to be transmitted is sufficient to be detected by PD 30; and that occurs long before change of the liquid solution would be perceived by the naked eye. That is, the invention readily manifests an incremental change in the color of the visible light being transmitted; so as to provide for accurate and prompt action to be taken, as needed.

The use of “spectroscopy technology analysis”, as taught by the present invention, also similarly diminishes the time for accurate quantitative analysis by utilizing using chemometrics. That is, by measurement of chemical data; and, more specifically, measuring an increasing presence of hydrogen-ions makes such chemometric measurement available promptly. That is hydrogen-ions in the NGM increase in response to increasing acidity; which enables chemometric measuring of resultant microbial-activity; such increasing acidity is the result of live bacteria, if any, surviving the microbial-biocidal treatment-cycle. The microbial activity exposure resulting from exposure incubation-conditions, increases the hydrogen-ions in the NGM; that increase is hydrogen-ions due to microbial-action, enables accurate evaluation, of a treatment-cycle failure to be made, within about three to about five hours of establishing culturing conditions.

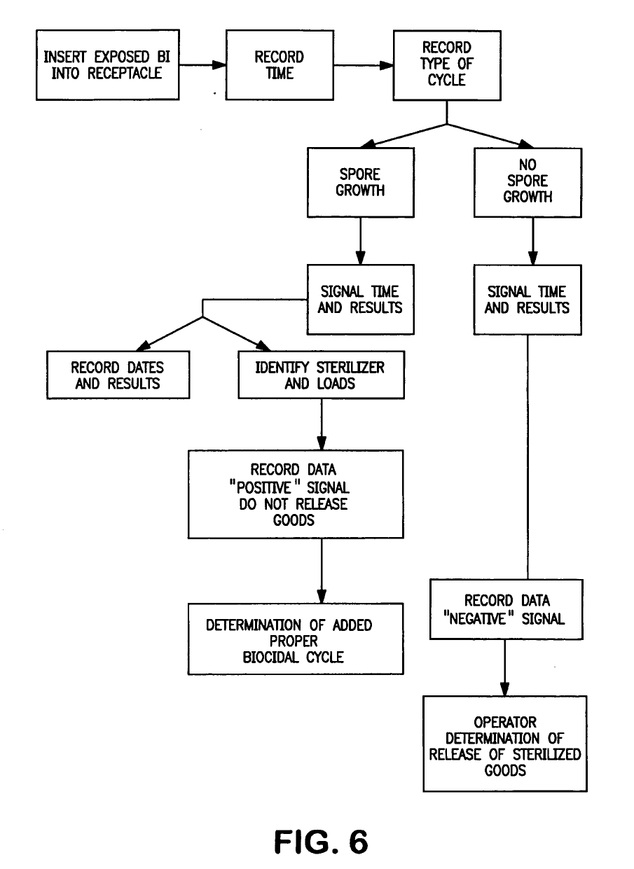

FIG. 5 and the schematic flow-chart presentation of FIG. 6, describe the sequence of steps in either the colormetric or spectroscopic technology radiant-energy analysis methods for expediting evaluation of a treatment-cycle. Such prompt determinations of microbial-activity are also visibly signaled by light 34 (FIG. 4) which blinks red; and, accurate documentation of data, including relevant times, take place and is recorded. Such early notification is particularly helpful for institutions, which may at times be forced to rely on “short interval” turn-around times, for certain goods, such as surgical instruments. Also, such prompt evaluations are particularly helpful in batch-food processing operations, by enabling prompt interruption of any ongoing food-processing operations, and preventing any commercial delivery when any in-line product fails as shown by the prompt testing results.

Enabling accurate determinations with decreased times for measurements, as available herein provide an important safety factor, in preventing possible harm to those relying on accurate measurements of sterility conditions. In addition, continuing incubation of a Test-Ampoule, beyond such decreased detection times, can provide an opportunity for further investigative analysis as to the cause, or causes, for failed treatment-cycle equipment or procedures.

The following tabulation sets forth some of the earlier discussed selections for differing cycles.

TABLE 1

| | | | |
|---|---|---|---|
| A. | Sterilizing Cycle | Thermal | ETO Phenol-Red |
| | (pH Indicator) | Bromocresol | |
| B. | Temperature of | 105° C. to 150° C. | 30° C. to 60° C. |
| | Cycle | | |
| C. | Spores | Geobacillus | Bacillus-atrophaeus |
| | | stearothermophilus | |
| D. | Incubation | 57° (+ or −2°) | 37° C. (+ or −2°) |
| E. | Yellow light | Approximate 588 | Peak Value 588 + 430 |
| | Wavelength in | nm using | nm; or other related |
| | Ångstroms | preselected pH | peak frequencies |
| F. | Minimum | 3 hrs. | 3 hrs. plus; dependent |
| | Incubation | | on rate of growth of |
| | Approximate Time | | Bacillus-atrophaeus |

A written description of the present invention, and of the manner and process for making and using it, including drawings and explanatory disclosures of functional methods and apparatus, for expediting detection of an inadequate biocidal-treatment cycle have been set forth so as to enable those, who become skilled in such newly-disclosed technology, to make and use the disclosed subject matter. However, the patentable scope of such invention is set forth in the claims; and the language of those claims is to be interpreted in the light of the above detailed description in conjunction with the accompanying drawings.

What is claimed is:

1. Process for automated-evaluation and authenticated documentation of effectiveness, or absence thereof, of selected individual microbial-biocidal treatment-cycles, comprising
A) providing a housing-structure, presenting
(i) individual test-cell receptacles, each for receiving an individual biological-indication (B-I) Test-Ampoule, for
(ii) evaluating, subsequent to intended exposure to a designated microbial-biocidal treatment-cycle,
(iii) biocidal effectiveness, or absence thereof, of such treatment-cycle on a B-I Test-Ampoule within an individual housing-structure test-cell;
B) correlating
(i) size and configuration of an individual B-I Test-Ampoule, with respect to
(ii) size and configuration of such an individual receptacle,
(iii) inserting such Test-Ampoule into such correlated size and configuration receptacle, for purposes of
C) biologically evaluating effectiveness following completion of such selected microbial-biocidal treatment-cycle, by
(i) automatically-controlling:
(a) heating within such housing-structure for establishing incubation conditions,
(b) establishing a selected incubation temperature within such B-I Test-Ampoule, and
(ii) recording time of establishment of incubation temperature, within
(iii) such correlated size and configuration B-I Test Ampoule, for
(a) evaluating intended microbial-biocidal results within such Test-Indicator; or, alternatively
(b) evaluating microbial-activity,
(iv) within such Test-Ampoule,

2. The invention of claim 1, including
D) presenting a plurality of test-cell receptacles, in one or more housing-structures, enabling
E) correlating respective sizes and configurations of selected individual test-cells, with respective individual B-I Test-Ampoules of selected differing sizes and configurations.

3. The invention of claim 2, including
F) controlling housing-structure heating means for automatically establishing and maintaining incubation temperature within a test-cell for its respective B-I Test-Ampoule, while
G) recording all relevant temperatures, times, and authenticating data, for
(i) supporting completion of test procedures, and
(ii) evaluating results of such procedures.

4. The invention of claim 3, further including
H) providing for a readily-observable alarm-type notice, to a user, of evaluation results, by selecting from the group consisting of:
(a) visible means,
(b) audible means, and
(c) combinations of (a) and (b).

5. The invention of claim 3, including
I) evaluating microbial-status, responsively to establishing incubation temperature within such a respective B-I Test-Indicator, by
J) utilizing radiant-energy for analyzing microbial-status following intended treatment-cycle exposure of such a B-I Test-Ampoule, by
(i) selecting radiant-energy analysis from the group consisting of
(a) colormetric-technology analysis, and
(b) spectroscopic-technology analysis, for
(ii) diminishing required time for evaluating microbial status within such B-I Test-Ampoule.

6. The invention of claim 5, including
(i) selecting colormetric-technology analysis utilizing radiant energy in a selected visible-light wavelength range, for enabling
(ii) evaluating presence of microbial-activity, if any, in such B-I Test Ampoule within about a three (3) to about a five (5) hour period, subsequent to establishment of such incubation temperature.

7. The invention of claim 5, including
(iii) selecting spectroscopic-technology analysis for chemometrically measuring increasing microbial-activity, if any, due to, growth of surviving microbes, enabling
(iv) evaluating presence of microbial-activity, if any, within about a three (3) to about a five (5) hour period, subsequent to establishing of such incubation temperature.

8. The invention of claim 6 utilizing colormetric technology, including
K) providing a liquid-Nutrient-Growth-Medium (NGM) within such a B-I Test-Ampoule, for initiating and sustaining microbial-activity during contact with microbes, if any, surviving such a treatment-cycle, including
L) selecting bacteria for such designated treatment-cycle, and
M) establishing a color for such NGM by selecting a pH responsive constituent, from the group consisting of
(a) Bromocresol Purple
(b) Phenol Red, and
(c) Bromothymol Blue, for
N) coloring such NGM, and
O) evaluating microbial activity, within such Test-Ampoule NGM, by
(i) detecting any change in NGM color, as established by such pH responsive-constituent for such NGM, as a result of
(ii) establishing culturing temperature within such Test-Ampoule,
(iii) causing an acidic reaction in such NGM due to microbial-activity.

9. The invention of claim 8, in which
P) expediting evaluation of microbial-action, if any, within such NGM is established, by
(i) selecting and positioning a light emitting diode (LED) for beaming a selected wavelength visible-light through such Test-Ampoule liquid NGM, with such beamed visible light,
(ii) having a color prevented from transmission due to the color established by selecting a pH indicator for NGM of such test-ampoule, and
(iii) detecting such beamed visible-light, if any, by photo-detector (PD) means, responding to incremental visible-light color, as
(iv) transmitted, if any,
(v) responsively to change of color of such NGM due to acidic effect of microbial-activity on such NGM within such Test-Ampoule.

10. Apparatus for evaluating microbial-status of one or more Biological-Indication (B-I) Test-Ampoules assembled to include selected bacteria, for analysis subsequent to exposure to a selected microbial-biocidal treatment-cycle, including
A) housing-structure means defining a selected number of individual receptacles, each presenting
(i) an interior configuration and size, capable of being correlated, with that of
(ii) a respective B-I Test-Ampoule to be monitored within such a receptacle, for evaluating biocidal effectiveness of intended exposure to such selected microbial-biocidal treatment-cycle;
B) controllable-heating means operable within such housing-structure, which are positioned
(i) contiguous to a selected test-cell for a respective B-I Test-Ampoule, for
(ii) establishing a culturing temperature for surviving bacteria, if any, as earlier selected, for
(iii) such B-I Test-Ampoule to be located within such correlated configuration and size test-cell;
C) means for detecting an indication of microbial-activity, if any, within such B-I Test-Ampoule responsive to establishment of such temperature for incubation of surviving microbes, if any, such selected Test-Ampoule as previously positioned;
D) control-means for recording timing of:
(i) placement of such a Test-Ampoule in its respective test-cell,
(ii) establishment of culturing temperature within such Test-Ampoule, and
(iii) evaluating microbial-action, if any, within such Test-Ampoule in response to establishing such culturing temperature.

11. The invention of claim 10, carried out on B-I Test Ampoules, selected from the group consisting of:
E) Test-Ampoules, with
(i) selected bacteria in contact with NGM during storage before exposure to such a treatment cycle and after such exposure; and
(ii) selected bacteria confined separately, within
(a) a capped polymeric external container, requiring
(b) rupture of a frangible, internally-disposed, sealed-capsule, for
(c) release of a liquid nutrient-growth-medium (NGM), for
(d) contact with surviving bacteria, if any, held within such tubular external-container,

12. Housing-structure, for use with a B-I Test-Ampoule of claim 11 which requires fracture of an internally-disposed sealed capsule, including
F) an opening in such housing-structure for such a Test-Ampoule, providing
(i) means for rupturing such internally-disposed sealed-capsule, for
(ii) initiating contact of such capsule-sealed NGM, with
(iii) surviving, bacteria, if any, as held within such external container of such Test-Ampoule, for
(iv) biocidal status evaluation of action of such NGM, by
(v) monitoring microbial change, if any, resulting from contact of such released NGM with any such surviving bacteria, following
(vi) timed exposure to culturing conditions within such external container.

13. The apparatus of claim 11 in which such evaluation of biocidal-status is monitored subsequent to exposure to a selected microbial-biocidal treatment-cycle, by use of
G) radiant-energy means, for expediting analysis of microbial-status, in which such means are selected from the group consisting of:
(a) colormetric-technology analysis measuring equipment, and
(b) spectroscopic-technology analysis measuring equipment.

14. The invention of claim 13, including
H) utilizing such colormetric-technology analysis equipment, subsequent to exposure to such microbial-biocidal treatment-cycle, by providing:
I) pH indicator means, during assembly of a B-I Test-Ampoule, which
(i) establishes pH of such liquid NGM, as well as:
(ii) coloring such liquid NGM, within such Test-Ampoule, by selecting
(iii) such pH indicator means from the group consisting of:
(a) Bromocresol Purple
(b) Phenol Red, and
(c) Bromothymol Blue;
J) visible-light emitting diode (LED) means and co-operating visible-light photo detector (PD) means, for evaluating status of a selected B-I Test Ampoule,
(i) positioned such LED means for projecting visible light of a color, having a wavelength other than the color established by such selected pH indicator means, for penetrating such NGM of such Test-Ampoule; with
(ii) such photo detector (PD) means, being positioned, to
(iii) detect incremental selected wavelength visible light, as transmitted, due to
(iv) change of transmittance of such NGM, responsive to:
(v) microbial-activity acidic change in such NGM,
(vi) due to microbial-action within such NGM, which produces
(vii) sufficient incremental transmission of such visible light, through such NGM, so as to enable
(viii) such photo-detector means (PD) to detect such incremental transmission of visible-length, within about three (3) to about five (5) hours after establishment of culturing temperature within such test-ampoule.

15. The invention of claim 13, utilizing
K) Bromocresol Purple, for
(i) control of pH, and
(ii) control of coloring of such NGM purple within such B-I Test-Ampoule, as selected for colormetric testing, to enable
(iii) such LED projected yellow-light, having a wavelength of about 588 nanometers (nm), being directed toward
(iv) such NGM, which previously prevented any measurable transmission of said yellow light, which, upon
(v) acidic change in such NGM within such test-ampoule being tested, provided by
(vi) microbial-activity, responsive to establishment of such culturing temperature enables transmitting of incremental yellow-light for detection, within about three (3) to about five (5) hours after initiating culturing temperature within such test-ampoule.

16. The invention of claim 13, including
L) selecting spectroscopic-technology analysis for evaluating bacterial-lethality results of such microbial-biocidal treatment cycle, by
(i) chemometric quantitative analysis of release of hydrogen-ions, if any, within such Test-Ampoule's liquid NGM, responsive to
(ii) increasing acidity within such liquid NGM due to microbial-activity resulting from establishing culturing-temperature within such test-ampoule.
